(19) United States
(12) Patent Application Publication
(10) Pub. No.: US 2005/0245899 A1
(43) Pub. Date: Nov. 3, 2005

(54) Dual purpose adapter

(76) Inventor: David Rork Swisher, St. Charles, MO (US)

Correspondence Address: Edward Scott Jarmolowicz, Esq.;Tyco Healthcare Group LP, 15 Hampshire Street, Mansfield, MA, 02048, US

(21) Appl. No.: 11/112,625

(22) Filed: Apr. 21, 2005

Related U.S. Application Data
(63) Continuation-in-part of application No. PCT/US03/34129, filed on Oct. 28, 2003.
(60) Provisional application No. 60/564,718, filed on Apr. 23, 2004.

(51) Int. Cl. A61M 25/18

(52) U.S. Cl. 604/533

(57) ABSTRACT

A connector is provided that can be used for both fluid aspirating applications and fluid introduction applications without a requirement for additional adapters. Direct connection to a vacuum tube or an enteral feeding connector can be possible without need of an additional rubber sleeve and/or an additional enteral feeding adapter. In one example, a first tubular portion has at least one protrusion along an exterior surface. A second tubular portion is also provided and coaxially located with respect to the first tubular portion. The second tubular portion has a first end within the first tubular portion and a second end external to the first tubular portion. The second tubular portion can have a stepped internal lumen forming the plurality of internal diameters and can be formed of a more pliable material than the first tubular portion. A method for forming a dual use adapter is also provided.

### REFERENCE TO RELATED APPLICATIONS

The present application is a continuation-in-part of PCT Application No. PCT/US2003/034129 filed on Oct. 28, 2003 by Shia, et al. and designating the United States of America and claims priority to and the benefit of U.S. Provisional Application Ser. No. 60/564,718 filed on Apr. 23, 2004 by Swisher. The entire contents of these applications are hereby incorporated by reference.

### TECHNICAL FIELD

The present application relates generally to an adapter for use with medical devices, and more particularly, to a dual purpose adapter that can alternately be used for coupling to a suction tube and an enteral feeding connector.

### BACKGROUND

Various medical systems are used for introducing and/or removing fluids from a patient. One such example involves nasogastric tubing, which is typically employed in hospitals, nursing homes and care facilities to remove fluids from the body of the subject, such as, for example, by aspirating fluids from a gastrointestinal tract (GI tract) of the subject, or to introduce fluids, such as, for example, nutrients, supplements and medicines, to the subject.

In an example of conventional use, a practitioner introduces the distal end of a flexible plastic nasogastric tube through a nasal canal of a subject via one of the nostrils. The distal end is passed through the pharynx and down the esophagus into the GI tract. The distal end can be passed into the duodenum, stomach, etc. depending on the particular application such as, for example, aspirating fluids, introduction for medication, feeding, etc. Several openings are formed in the distal end that permits passage of gastric fluids, nutrients, medication and the like.

The proximal end of the nasogastric tube exits the nostril and may be connected to the suction source or a feeding pump through a connector. In a fluid aspirating application, stomach fluids are drawn through the openings in the distal end, through the passageway and into the collection vessel, as facilitated by the suction source. In a fluid introduction application, nutrients, medication, etc. are injected into the passageway and forced through the openings in the distal end and into for example, the duodenum.

The connector is connected to a second tube that is connected to the suction source, or alternatively, to a feeding pump. Frequently, the nasogastric tubing must be alternated to a source for suction, feeding or introduction of an injection. To alternate the nasogastric tubing application, the second tube is removed from the connector or the connector is removed from the proximal end of the nasogastric tubing and the desired connection is made.

The valve systems of the prior art are designed to connect to several different types of tubes. For example, one tube connection is fixed to a sump-tube (nasal or gastric), one is dedicated for medication and another is for a suction line or adapter for enteral feeding. As shown in FIG. 1, a valve 10 of the prior art can be used to provide selective connections between a stomach or nasal tube 12, an irrigation device 14 and a rigid connector 16. The rigid connector 16 can be used to connect to an enteral feeding source or vacuum source. A rubber sleeve 15 is typically required to be added over the rigid connector 16 for connection to an enteral adapter 20 when continuous feeding is required by the user. However, it is common that the sleeve 15 would have been typically discarded or misplaced making that action impossible. Alternatively, another rubber sleeve adapter 25 is required to connect a suction line 30 to the rigid connector 16.

The adapter 20 for enteral feeding is typically loosely packaged in the initial sterile packaging and is often discarded upon the initial use or opening of the valves of the prior art. Some prior art valves include an adapter for enteral feeding that is tethered via a ring thus keeping it with the valve system. However, this tethered adapter becomes problematic in that it is obtrusive and gets in the way of the user or clinician.

### SUMMARY

While not limited to use with nasogastric tubing or valves, the present invention is directed toward the need for a connector that can be used for both fluid aspirating applications and fluid introduction applications without a requirement for additional adapters. Embodiments of the present invention provide a dual use adapter that can connect to a vacuum tube or an enteral feeding connector without need of an additional rubber sleeve and/or an additional enteral feeding adapter.

In one embodiment, the present invention provides a dual use adapter having a first tubular portion having at least one protrusion along an exterior surface. A second tubular portion is also provided and coaxially located with respect to the first tubular portion. The second tubular portion has a first end within the first tubular portion and a second end external to the first tubular portion. The second tubular portion has a stepped internal lumen forming the plurality of internal diameters and is formed of a more pliable material than the first tubular portion.

In an alternative embodiment, the dual use adapter includes a first tubular portion and a second tubular portion. The second tubular portion has a first end within the first tubular portion and a second end external to the first tubular portion. The second tubular portion that is external to the first tubular portion has a tapered exterior diameter decreasing in diameter approaching a distal end of the second end and a tapered interior diameter increasing in diameter approaching the distal end of the second end. In this embodiment, the first tubular portion also has at least one protrusion along an exterior surface, and the second tubular portion is coaxially located with respect to the first tubular portion.

In a further embodiment the dual use adapter is provided with a receiving end configured to mount within a vacuum tube. A first tubular portion has at least one circumferential protrusion along an exterior surface. The second tubular portion is coaxially located with respect to the first tubular portion and has an end within the first tubular portion and an end external to the first tubular portion at the receiving end of the dual use adapter. The second tubular portion is formed of a more pliable material than the first tubular portion.

In yet a further embodiment of the present invention is a method of forming a dual use adapter. The method includes the acts of providing a first tubular portion with at least one protrusion along an exterior surface and having a first interior lumen and providing a second tubular portion formed of a more pliable material than the first tubular portion and having a stepped internal lumen forming a plurality of internal diameters. The method also includes affixing a second tubular portion partially into the first tubular portion. The second tubular portion has a first end within the first tubular portion and a second end external to the first tubular portion.

### BRIEF DESCRIPTION OF THE DRAWINGS

The invention will be apparent from the description herein and the accompanying drawings, in which like reference characters refer to the same parts throughout the different views.

FIG. 1 is a perspective view of the various adapters for use in a valve system of the prior art;

FIG. 2 is a perspective view of an adapter according to an embodiment of the invention;

FIG. 3 is a cross-sectional view of the adapter of FIG. 2;

FIG. 4 is a cross-sectional view of the adapter of FIG. 2 with an enteral feeding connector inserted therein;

FIG. 5 is a cross-sectional view of the second tubular portion of FIG. 2;

FIG. 6 is a cross-sectional view of another adapter according to an embodiment of the invention;

FIG. 7 is a cross-sectional view of another adapter according to an embodiment of the invention; and

FIG. 8 is a perspective view of a valve including an adapter according to an embodiment of the invention.

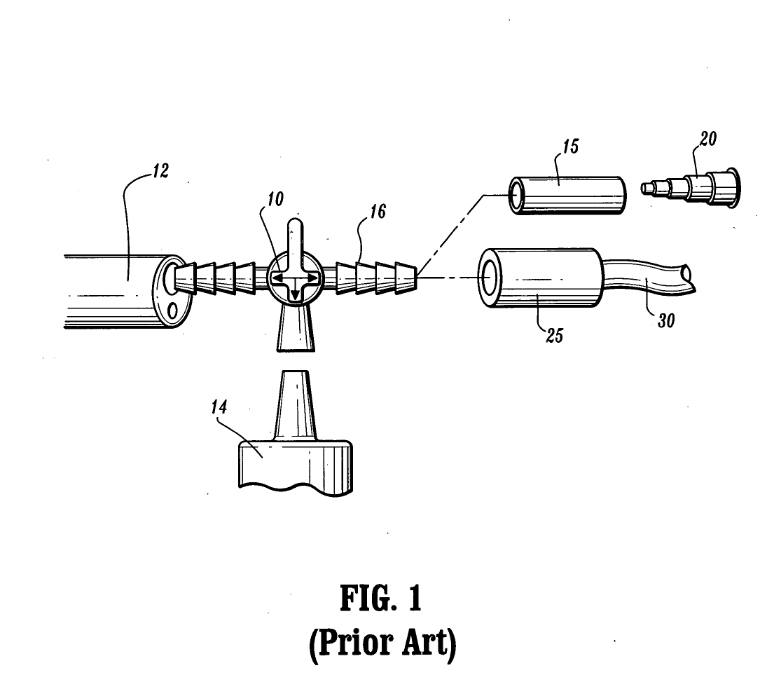

### DETAILED DESCRIPTION

The present invention can offer a connector that can be used for both fluid aspirating applications and fluid introduction applications without a requirement for additional adapters such as, for example, a tethered adapter which is provided with some conventional connectors. The present invention can also reduce a need for additional ports to accommodate the multiple types of connections that may be needed. The present invention may also be used in conjunction with a valve.

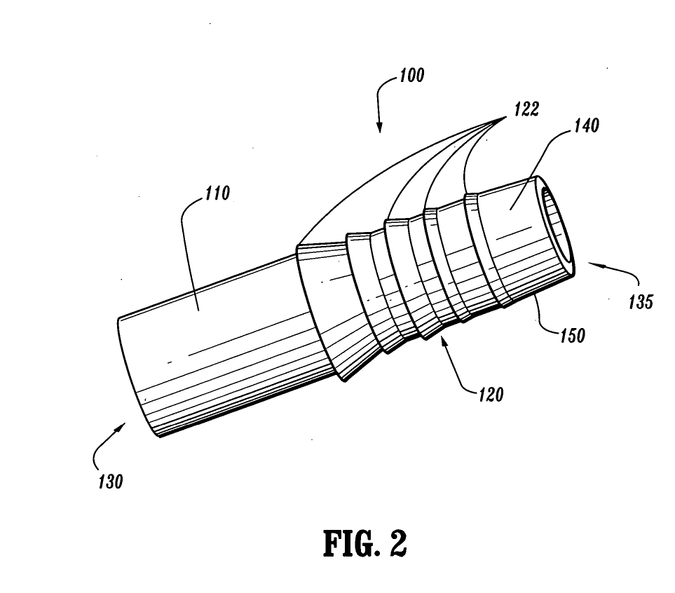

According to an embodiment of the invention as illustrated by way of example in FIG. 2, a dual use adapter 100 is provided having a proximal end 130 and a distal end 135. The adapter 100 has a first portion 110 having an external surface 120 for mating with an internal diameter of a tube, such as, for example, a vacuum tube. The external surface 120 is configured with at least one protrusion 122 to aid in retaining the tube sized to mate with the adapter 100. The first portion 110 may be rigid and the protrusions 122 may include external barbs or other friction-enhancing surface features on the external surface 120 that are compatible with most suction tube adapters for connecting to a vacuum source. The protrusions 122 may extend circumferentially along an outer diameter, or may be located along only a portion of the exterior of the first portion 110.

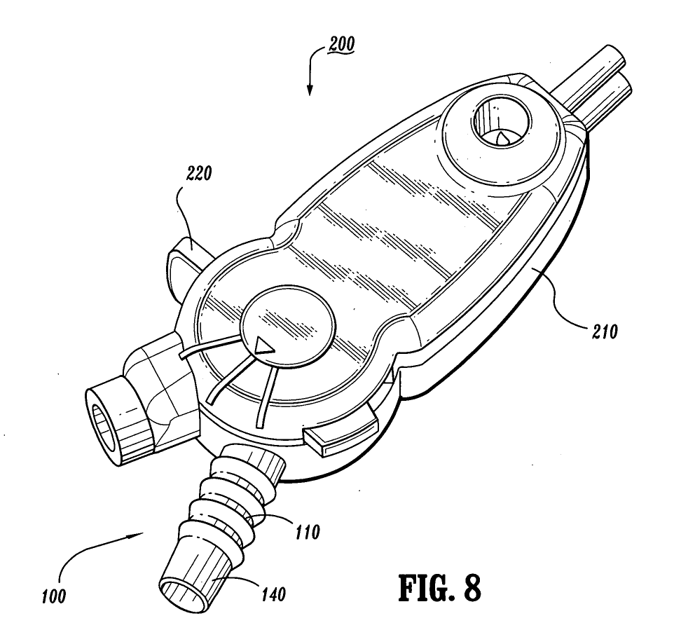

The proximal end 130 of the adapter 100 may be coupled to a patient or other subject or medical device, such as by affixing the proximal end 130 to the medical device, tubing, a valve or other fluid handling device. In one example, the first portion 110 can be integrally formed with or affixed to a valve housing (FIG. 8). The distal end 135 of the adapter 100 is configured to form a receiving end for a connection by receiving the tubing or other medical device.

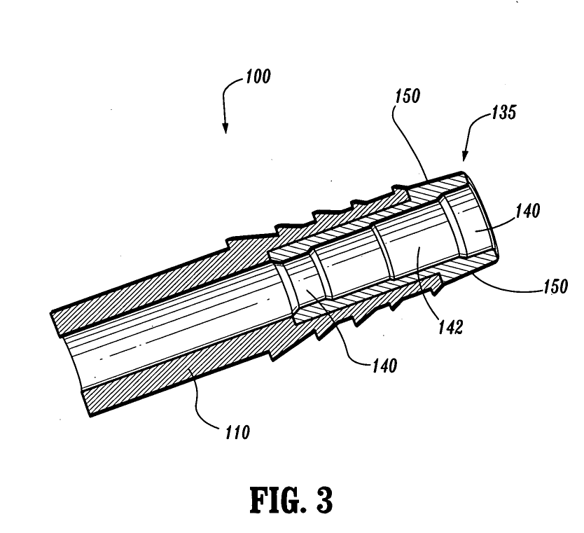

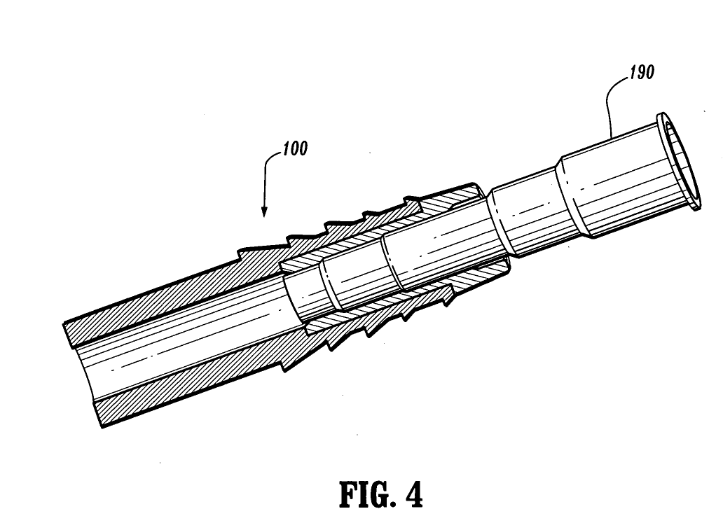

A second tubular portion 140 is located at the distal end 135 of the adapter 100. As shown in the cross-sectional view of FIG. 3, a portion of the second tubular portion 140 is located within the first portion 110. The second tubular portion 140 has an internal surface 142 for mating with an external surface of a tube or connector. The second tubular portion 140 can be a softer, e.g., more pliable, component that the first portion 110. An internal diameter of the second tubular portion 140 may be variable, such as by the use of one or more tapers and/or steps. In the illustrated example, the internal diameter varies to mate with the external diameters of an enteral feeding connector 190, shown by way of example in FIG. 4. An external surface 150 of the second tubular portion 140 extending beyond the first tubular portion 110 may also be tapered to aid in inserting the adapter 100 into a tube.

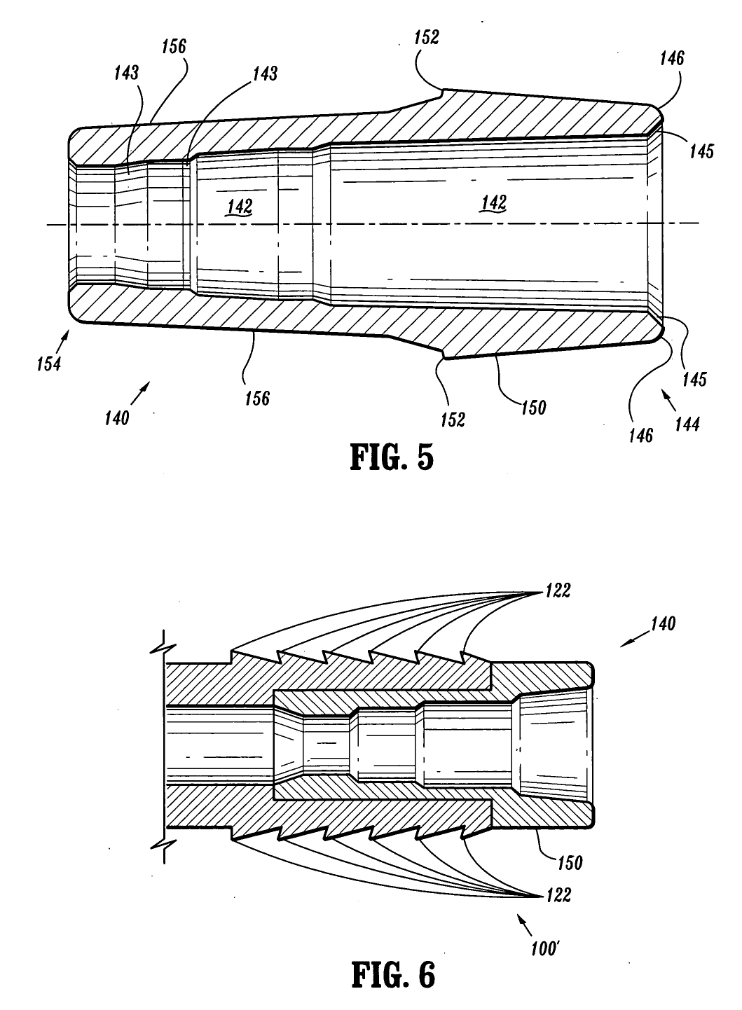

FIG. 5 illustrates a cross-sectional view of an example of the second tubular portion 140. In this example, the interior surface 142 has a stepped internal diameter that is tapered between steps 143. A distal end 144 of the second tubular portion includes a chamfered interior leading edge 145 and a rounded exterior leading edge 146. A tapered exterior diameter is also provided along an exterior surface 150 leading to a mounting flange 152 that abuts the first tubular portion 110. The mounting flange 152 may transmit force applied to the second tubular portion 140 to the first tubular portion 110. A proximal end 154 of the second tubular portion 140 may also have rounded corners and may transmit force applied to the second tubular portion 140 to the first tubular portion 110. Surface friction between the mounting surface 156 and the corresponding interior surface of the first tubular portion 110 may also transmit force applied to the second tubular portion 140 to the first tubular portion 110.

The second tubular portion 140 may be formed of a rubber-like substance and may be bonded, insert molded, co-molded into or integrally formed with the first tubular portion 110 to be compatible with a variety of smaller enteral feeding connectors. In one example, the adapter 100 may be used in situations where suction is not applied while a user is offering continuous feeding via an enteral feeding system. For example, a valve product where there is continuous suction or intermittent suction for a short period of time, but then goes to continuous feeding when the user is finished with the suction process.

The first tubular portion may be formed of a wide variety of materials. In one example, the first tubular portion 110 is formed of polycarbonate. The second tubular portion may also be formed of a wide variety of materials, one example of which is polyvinyl chloride (PVC). Optionally, the first tubular portion and second tubular portion may be formed of different colors.

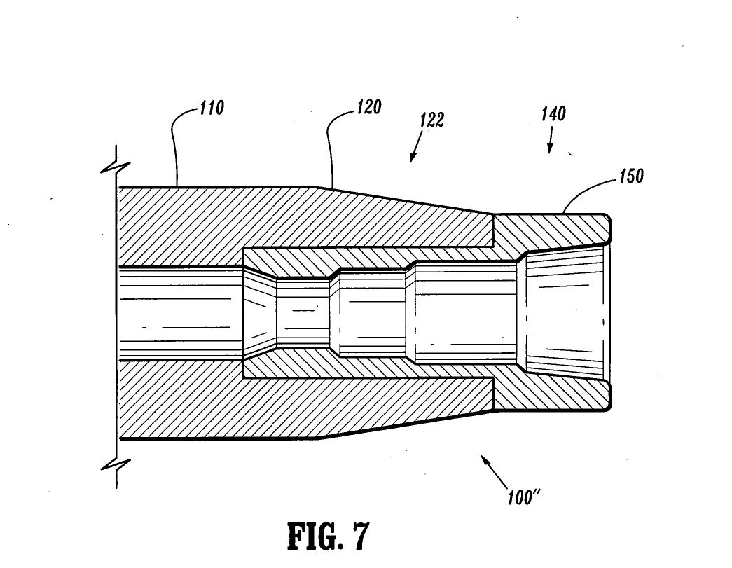

FIG. 6 is a cross-sectional view of another example of an adapter 100′ having a second tubular portion 140 without a tapering exterior diameter along its exterior surface 150. In this example, the protrusions 122 are in the form of barbs. FIG. 7 provides a further cross-sectional illustration of another example of an adapter 100″ having a taper forming the protrusion 122 along the exterior surface 120 of the first tubular portion 140.

FIG. 8 illustrates an example of a valve 200 having a valve housing 210 integrally formed with a first tubular portion 110 of the adapter 100. The valve 200 also includes a selection knob 220 for actuation of the valve 200. It is understood that the present invention may be used with a wide variety of valves and fluid handling devices.

In another embodiment, the present invention may include a method for forming a dual use adapter. The method may include the acts of providing a first tubular portion with at least one protrusion along an exterior surface and a first interior lumen. Providing a second tubular portion formed of a more pliable material than the first tubular portion and having a stepped internal lumen forming a plurality of internal diameters. A further act of affixing the second tubular portion partially into the first tubular portion is also included. The second tubular portion has a first end within the first tubular portion and a second end external to the first tubular portion. The method may optionally, in the act of affixing, include the acts of bonding, insert molding, co-molding or integrally forming. An additional optional step includes providing a valve housing coupled to the first tubular portion and in communication with the first interior lumen.

The present invention has been described by way of example, and modifications and variations of the described embodiments will suggest themselves to skilled artisans in this field without departing from the spirit of the invention. Aspects and characteristics of the above-described embodiments may be used in combination. The described embodiments are merely illustrative and should not be considered restrictive in any way. The scope of the invention is to be measured by the appended claims, rather than the preceding description, and all variations and equivalents that fall within the range of the claims are intended to be embraced therein.

1. A dual use adapter, comprising:
a first tubular portion having at least one protrusion along an exterior surface; and
a second tubular portion, coaxially located with respect to the first tubular portion, having a first end within the first tubular portion and a second end external to the first tubular portion and having a stepped internal lumen forming the plurality of internal diameters;
the second tubular portion formed of a more pliable material than the first tubular portion.

2. The dual use adapter of claim 1, wherein at least two of the plurality of internal diameters mate with a plurality of external diameters of an enteral feeding connector.

3. The dual use adapter of claim 1, the second tubular portion having internal diameters tapered between steps.

4. The dual use adapter of claim 1, the first tubular portion having a plurality of circumferential protrusions along an exterior surface, configured to retain a tube fitted over the circumference of the first tubular portion.

5. The dual use adapter of claim 1, the first tubular portion having a plurality of circumferential protrusions along an exterior surface, at least two of the circumferential protrusions having different outside diameters.

6. The dual use adapter of claim 1, the second tubular portion having a tapered exterior diameter of the second end, decreasing in diameter approaching a distal end of the second end.

7. The dual use adapter of claim 6, the second tubular portion having a tapered interior diameter of the second end, increasing in diameter approaching the distal end of the second end.

8. The dual use adapter of claim 7, the distal end of the second end of the second tubular portion having a rounded outside edge and a chamfered inside edge.

9. The dual use adapter of claim 1, the at least one protrusion along an exterior surface being a circumferential protrusion.

10. The dual use adapter of claim 1, the at least one protrusion along an exterior surface being a tapered external diameter, increasing in diameter away from a location at which the first end of the second tubular portion meets the second end of the second tubular portion.

11. The dual use adapter of claim 1, the at least one protrusion along an exterior surface being at least one barb.

12. The dual use adapter of claim 1, the first tubular portion being formed of polycarbonate.

13. The dual use adapter of claim 1, the second tubular portion being formed of polyvinyl chloride.

14. The dual use adapter of claim 1, the first tubular portion having a first color and the second tubular portion having a second color different from the first color.

15. The dual use adapter of claim 1, the first tubular portion being integral with a valve housing.

16. A dual use adapter, comprising:
a first tubular portion having at least one protrusion along an exterior surface; and
a second tubular portion, coaxially located with respect to the first tubular portion, having a first end within the first tubular portion and a second end external to the first tubular portion, the second end having a tapered exterior diameter decreasing in diameter approaching a distal end of the second end, and the second end having a tapered interior diameter increasing in diameter approaching the distal end of the second end.

17. The dual use adapter of claim 16, the second tubular portion formed of a more pliable material than the first tubular portion.

18. The dual use adapter of claim 16, the distal end of the second end of the second tubular portion having a rounded outside edge and a chamfered inside edge.

19. The dual use adapter of claim 16, the second tubular portion having a stepped internal lumen forming the plurality of internal diameters.

20. The dual use adapter of claim 16, the at least one protrusion along an exterior surface being at least one barb.

21. The dual use adapter of claim 16, the first tubular portion being integral with a valve housing.

22. A dual use adapter having a receiving end, comprising:
a first tubular portion having at least one circumferential protrusion along an exterior surface; and
a second tubular portion, coaxially located with respect to the first tubular portion, having a first end within the first tubular portion and a second end external to the first tubular portion at the receiving end of the dual use adapter;
the second tubular portion formed of a more pliable material than the first tubular portion;
the receiving end configured to mount within a vacuum tube.

23. The dual use adapter of claim 22, the second tubular portion having a tapered interior diameter of the second end, increasing in diameter approaching the distal end of the second end, the interior diameter including a stepped interior diameter, decreasing in diameter approaching the first end of the second tubular portion.

24. The dual use adapter of claim 22, further comprising a valve housing located at an end of the first tubular portion opposite the receiving end of the dual use adapter.

25. A method of forming a dual use adapter, comprising the steps of:
providing a first tubular portion with at least one protrusion along an exterior surface and having a first interior lumen;
providing a second tubular portion formed of a more pliable material than the first tubular portion and having a stepped internal lumen forming a plurality of internal diameters; and
affixing the second tubular portion partially into the first tubular portion, the second tubular portion having a first end within the first tubular portion and a second end external to the first tubular portion.

26. The method of claim 25, the act of affixing comprising bonding.

27. The method of claim 25, the act of affixing comprising insert molding.

28. The method of claim 25, the act of affixing comprising co-molding.

29. The method of claim 25, further comprising providing a valve housing coupled to the first tubular portion and in communication with the first interior lumen.
